(19) United States
(12) Patent Application Publication
(10) Pub. No.: US 2021/0208044 A1
(43) Pub. Date: Jul. 8, 2021

(54) METHOD

(71) Applicant: Conopco, Inc., d/b/a UNILEVER, Englewood Cliffs, NJ (US)

(72) Inventors: Lynsey Joanne COAN, Wirral (GB); Colin Christopher David GILES, Oxton, Wirral (GB); Jennifer Amy GLENDAY, Sheffield (GB); Raquel GUTIERREZ-ABAD, Stockport (GB); Matias LUCK, Wirral (GB)

(21) Appl. No.: 17/056,535

(22) PCT Filed: May 29, 2019
(86) PCT No.: PCT/EP2019/063982

§ 371 Date: Nov. 18, 2020

(30) Foreign Application Priority Data
Jun. 7, 2018 (EP) 18176639.5

(51) Int. Cl. G01N 11/00; G01N 1/38; A61K 8/04; A61Q 5/12; A61K 8/81

(52) U.S. Cl. CPC G01N 11/00; G01N 1/38; A61K 2800/5424; A61Q 5/12; A61K 8/8152; A61K 8/042

(57) ABSTRACT

A method of predicting rinse properties of a composition from a surface, comprising the steps of: I. providing a neat treatment composition; II. preparing a series of aqueous dilutions of the neat treatment composition; III. measuring the viscosities of the neat treatment composition and the aqueous dilutions of the treatment composition using a suitable method such as a Brookfield viscometer fitted with a T-B spindle and Helipath, at 0.5 rpm and 25° C.; IV. correlating the measured viscosities to the rinse properties of the neat treatment composition; and v. optionally correlating the rinse properties of the neat treatment composition to the amount of water used to rinse the neat composition from a surface.

### FIELD OF INVENTION

The present invention relates to method for determining rinse properties of compositions, having particular application in the field of hair care.

### BACKGROUND AND PRIOR ART

Many products, formulated for use on surfaces, are designed to be rinsed off during use. Such products include shampoos and conditioning compositions for use on hair. These may be used as part of a hair care regime such as a daily wash and care process. These products often deposit benefit agents, for example silicones, onto the hair surface. Other, leave on, compositions deposit benefit agents onto hair that remain on the hair until the hair is next washed.

The rinsing of a composition from a surface is an important phenomenon. It can affect the way a consumer perceives product performance or makes the decision about whether to stop or continue rinsing. Rinsing properties of hair treatment compositions affect the length of time that a consumer rinses his/her hair and so directly influence, ipso facto, the amount of water that a consumer uses when using a rinse-off product.

We have found that when a consumer rinses conditioner from his/her hair, he/she will stop rinsing when a satisfactory constant level of smooth feel is reached (also referred to herein as the “rinsed friction plateau”). Compositions that are formulated to enable the consumer to reach his/her rinsed friction plateau sooner, thus cause him/her to stop rinsing thus preventing further consumption of water.

Despite the prior art there remains a need for a method for determining rinse properties of compositions that is reliable and accessible and that can be quickly and easily carried out.

We have found that when a conditioning gel phase composition is applied to hair during a wash/care process, the gel phase is deposited onto the hair surface. When the deposited gel phase comes into contact with water (during a rinse step), the structure of the gel phase must be broken up in order for it to be efficiently removed from the hair. The greater the disruption to the gel phase, the easier and faster it is removed and, ipso facto, the less water is required to complete the rinse.

Viscosity is a key property of a cosmetic composition and is determined by its rheological structure. If the structure is disrupted, then the viscosity is reduced. We have found that rinsing properties of cosmetic compositions are related to changes that occur to the viscosity upon contact with water, such that as the viscosity reduces, the rate of rinsing increases.

We have found that when a cosmetic composition is applied to a surface, its rheological structure is high. When water is added, in a rinse process, the structure begins to breakdown, the composition becomes less substantive to the surface, causing it to be removed from the surface. As the breakdown progresses, the rate of removal from the surface increases.

We have further found that the disruption to the rheological structure, for example the gel phase can be measured by a reduction in its viscosity that occurs upon dilution with water. For any given quantity of water, the extent of viscosity reduction is directly related to how quickly and easily it will be removed from the surface. The amount of water required to rinse a cosmetic composition from a surface is, therefore, directly related to the rate of viscosity reduction of the composition upon contact with water.

A method based on these findings provides a reliable and accessible way of predicting rinse properties of compositions.

### STATEMENT OF INVENTION

In a first aspect, the invention provides a method of predicting rinse properties of a composition from a surface, comprising the steps of:
i. providing a neat cosmetic treatment composition;
ii. preparing a series of aqueous dilutions of the neat treatment composition;
iii. measuring the viscosities of the neat treatment composition and the aqueous dilutions of the treatment composition using a suitable method such as a Brookfield viscometer fitted with a T-B spindle and Helipath, at 0.5 rpm and 25° C.;
iv. correlating the measured viscosities to the rinse properties of the neat treatment composition;
v. optionally correlating the rinse properties of the neat treatment composition to the amount of water used to rinse the neat composition from a surface.

General Description of the Invention

The Method

The method of the invention measures the viscosities of a composition, which is related to the rinse properties of the composition. The rinse properties are related to the quantity of water required to rinse the composition from a surface.

The composition is a cosmetic composition. A cosmetic composition, for example, a personal care composition, is intended for application to the human body, particularly the skin or hair. Preferably the composition is selected from a hair composition (for example a hair cleansing composition, a hair conditioning composition or a hair styling composition) and a skin composition (for example, a skin cleansing composition or a skin conditioning composition).

A preferred method of the invention comprises the steps of:
i) providing a neat hair treatment composition;
ii) preparing a series of aqueous dilutions of the neat hair treatment composition;
iii) measuring the viscosities of the neat hair treatment composition and the aqueous dilutions of the treatment composition;
iv) correlating the measured viscosities to the rinse properties of the neat hair treatment composition; and
v) optionally correlating the rinse properties of the neat hair treatment composition to the amount of water used to rinse the neat hair composition from a hair surface.

Advantageously, the method of the invention may be used to compare the viscosities and, therefore, the rinse properties of different compositions, for example a composition before and after a modification to the composition has been carried out. This is accomplished by carrying out the method using a first neat treatment composition and then carrying out the method using a second neat treatment composition.

Preferably, the method includes repeating steps (i) to (iv) for a second neat treatment composition and comparing the viscosities of the first and second neat treatment compositions to determine the relative rate of rinsing of the first and second neat treatment compositions. The composition having the greater reduction in viscosity on dilution will be rinsed faster from the surface.

Preferably the method includes the step of comparing the first and second neat treatment compositions and correlating the viscosity and/or rate of rinsing of the compositions to the amount of water used to rinse the neat composition from a surface. The composition having the greater reduction in viscosity on dilution, or the greater rate of rinsing, will require less water to be rinsed from the surface.

The Composition

The composition is preferably formulated as a rinse off composition.

Preferably, the composition is structured. By structured is meant its molecular orientation forms a gel phase or a lamellar phase.

The composition is preferably a hair treatment composition.

Rinse off hair treatment compositions for use in the present invention are preferably selected from a shampoo and a conditioner, most preferably a conditioner.

Compositions for use in the method of the invention are preferably formulated as conditioners for the treatment of hair (typically after shampooing) and subsequent rinsing.

Preferred conditioners comprise a conditioning base. The conditioning base preferably forms a gel phase.

Treatments compositions for use in the method of the current invention preferably comprise conditioning agents. Conditioning agents are preferably selected from cationic surfactants, used singly or in admixture.

Cationic surfactants useful in compositions for use in the method of the invention contain amino or quaternary ammonium hydrophilic moieties which are positively charged when dissolved in aqueous composition.

Examples of suitable cationic surfactants are those corresponding to the formula

[N(R₁)(R₂)(R₃)(R₄)]⁺(X)⁻

in which R₁, R₂, R₃ and R₄ are independently selected from (a) an aliphatic group of from 1 to 22 carbon atoms, or (b) an aromatic, alkoxy, polyoxyalkylene, alkylamido, hydroxyalkyl, aryl or alklaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

The most preferred cationic surfactants for compositions for use in the method of the present invention are monoalkyl quarternary ammonium compounds in which the akyl chain lengthy is C₈ to C₁₄.

Suitable examples of such materials correspond to the formula

[N(R₅)(R₆)(R₇)(R₈)]⁺(X)⁻

in which R₅ is a hydrocarbon chain having 8 to 14 carbon atoms or a functionalised hydrocarbyl chain with 8 to 14 carbon atoms and containing ether, ester, amido or amino moieties present as substituents or as linkages in the radical chain, and R₆, R₇ and R₈ are independently selected from (a) hydrocarbyl cahins of from 1 to about 4 carbon atoms, or (b) functionalised hydrocarbyl chains having from 1 to about 4 carbon atoms and containing one or more aromatic, ether, ester, amido or amino moieties present as substituents or as linkages in the radical chain, and X is a salt-forming anion such as those selected from halogen, (e.g. chloride, bromide), acetate, citrate, lactate, glycolate, phosphate nitrate, sulphate and alkylsulphate radicals.

The functionalised hydrocarbyl chains (b) may suitably contain one or more hydrophilic moieties selected from alkoxy (preferably C₁-C₃ alkoxy), polyoxyalkylene, alkylester, and combinations thereof.

Preferably the hydrocarbon chains R₁ have 12 to 14 carbon atoms, most preferably 12 carbon atoms. They may be derived from source oils which contain substantial amounts of fatty acids having the desired hydrocarbyl chain length. For example, the fatty acids from palm kernel oil or coconut oil can be used as a source of C₈ to C₁₂ hydrocarbyl chains.

Typical monoalkyl quarternary ammonium compounds of the above general formula for use in compositions for use in the method of the invention include:
(i) Lauryl trimethylammonium chloride (available commercially as Arquad C35 ex Akzo); cocodimethyl benzyl ammonium chloride (available commercially as Arquad DMCB-80 ex-Akzo)
(ii) Compounds of the formula:

[N(R₁)(R₂)((CH₂CH₂O)ₓH)((CH₂CH₂O)_{y}H]⁺(X)⁻

wherein:

x+y is an integer from 2 to 20;

R₁ is a hydrocarbyl chain having 8 to 14, preferably 12 to 14, most preferably 12 carbon atoms and containing ether, ester, amido or amino moieties present as substituent's or as linkages in the radical chain;

R₂ is a C₁-C₃ alkyl group or benzyl group, preferably methyl, and

X is a salt-forming anion such as those selected from halogen, (e.g. chloride, bromide), acetate, citrate, lactate, glycolate, phosphate nitrate, sulphate, methosulphate and alkylsulphate radicals.

Suitable examples are PEG-n lauryl ammonium chlorides (where n is the PEG chain length), such as PEG-2 cocomonium chloride (available commercially as Ethoquad C12 ex-Akzo Nobel); PEG-2 cocobenzyl ammonium chloride (available commercially as Ethoquad CB12 ex-Akzo Nobel); PEG-5 cocomonium methosulphate (available commercially as Rewoquat CPEM ex Rewo); PEG-15 cocomonium chloride (available commercially as Ethoquad C/25 ex-Akzo).
(iii) Compounds of the formula:

[N(R₁)(R₂)(R₃)((CH₂)ₙOH)]⁺(X)⁻

wherein:

n is an integer from 1 to 4, preferably 2;

R₁ is a hydrocarbyl chain having 8 to 14, preferably 12 to 14, most preferably 12 carbon atoms;

R₂ and R₃ are independently selected from C₁-C₃ alkyl groups, and are preferably methyl, and

X- is a salt-forming anion such as those selected from halogen, (e.g. chloride, bromide), acetate, citrate, lactate, glycolate, phosphate nitrate, sulphate, alkylsulphate radicals. Suitable examples are lauryldimethylhydroxyethylammonium chloride (available commercially as Prapagen HY ex-Clariant).

Mixtures of any of the foregoing cationic surfactants compounds may also be suitable.

Examples of suitable cationic surfactants for use in hair compositions for use in the method of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant is cetyltrimethylammonium chloride, available commercially, for example as DEHYQUART, ex Henkel.

The level of cationic surfactant is preferably from 0.01 to 10, more preferably 0.05 to 5, most preferably 0.1 to 2 w.t. % of the total composition.

A preferred conditioner comprises a conditioning gel phase. Such conditioners and methods for making them are described in WO2014/016354, WO2014/016353, WO2012/016352 and WO2014/016351.

The conditioning compositions may also comprise other optional ingredients. Such ingredients include, but are not limited to; fatty material, deposition polymers and further conditioning agents.

Conditioner compositions preferably additionally comprise fatty materials. The combined use of fatty materials and cationic surfactants in conditioning compositions is believed to be especially advantageous, because this leads to the formation of a structured lamellar or liquid crystal phase, in which the cationic surfactant is dispersed.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Examples of suitable fatty alcohols include cetyl alcohol, stearyl alcohol and mixtures thereof. The use of these materials is also advantageous in that they contribute to the overall conditioning properties of compositions.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof. The level of fatty material in conditioners is suitably from 0.01 to 15, preferably from 0.1 to 10, and more preferably from 0.1 to 5 percent by weight of the total composition. The weight ratio of cationic surfactant to fatty alcohol is suitably from 10:1 to 1:10, preferably from 4:1 to 1:8, optimally from 1:1 to 1:7, for example 1:3.

Further conditioning ingredients include esters of fatty alcohol and fatty acids, such as cetyl palmitate.

A conditioning composition for use in the present invention may preferably comprise a miscellar structured liquid.

The pH of a conditioner comprising the present composition is preferably 3-5. More preferably the pH of the composition is 4.5-5.5.

A Viscosity Reduction Agent

Preferably, the method of the invention includes a step of adding a viscosity reduction agent to the neat treatment composition to reduce the viscosity.

A preferred viscosity reduction agent is a hydrophobically modified anionic polymer Preferably, the hydrophobically modified anionic polymer is an acrylate or methacrylate polymer.

Preferably, the hydrophobic modification comprises alkylation. Preferably, the alkyl group comprises from 6 to 30 carbons, more preferably from C12 to C30, even more preferably from 16 to 28 and most preferably from 18 to 24 carbons.

A preferred polymer is sold by Rohm & Haas under the tradename Aculyn, the most preferred of which is Aculyn 28™.

The polymer is preferably added at a level of from 0.01 to 5 wt %, more preferably from 0.02 to 0 5 wt %, even more preferably from 0.03 to 4 wt % and most preferably from 0.05 to 4 wt %, by total weight of the hair treatment composition.

Preferably, the method of the invention includes an additional step of measuring the viscosity before and after the addition of the viscosity reduction agent.

The aqueous dilutions

Preferably, at least 2 dilutions are used, more preferably from 2 to 8 dilutions are used. Preferably a 1 in 2 dilution and a 1 in 4 dilution are used.

The diluted compositions are preferably prepared by mixing the neat composition with water to the desired level of dilution.

Preferably, water is added to neat composition in small amounts with mixing after addition of each amount.

The speed of water addition and the amount and speed of mixing should be consistent for a series of diluted compositions.

Preferably, the dilution is allowed to equilibrate, for example by standing, for example for one hour, before the viscosity is measured.

Where two or more compositions to be compared according to the method of the invention, consistent mixing and speed of water addition should be adhered to for each composition.

The Viscosity Measurement

Any suitable method of measuring the viscosity of the neat composition and the diluted compositions can be used. For example, using a suitable method such as a Brookfield viscometer fitted with a T-B spindle and Helipath, at 0.5 rpm and 25° C.

Correlating the Measured Viscosities

We have found that when a conditioning gel phase composition is applied to hair during a wash/care process, the gel phase is deposited onto the hair surface. When the deposited gel phase comes into contact with water (during a rinse step), the structure of the gel phase must be broken up in order for it to be efficiently removed from the hair. The greater the disruption to the gel phase, the easier and faster it is removed and, ipso facto, the less water is required to complete the rinse.

Viscosity is a key property of a cosmetic composition and is determined by its rheological structure. If the structure is disrupted, then the viscosity reduces. We have found that rinsing properties of cosmetic compositions are influenced by changes that occur to the viscosity upon contact with water.

We have found that when a cosmetic composition is applied to a surface, its rheological structure is high. When water is added, in a rinse process, the structure begins to breakdown, the composition becomes less substantive to the surface, causing it to be removed from the surface. As the breakdown progresses, the rate of removal from the surface increases.

We have further found that the disruption to the rheological structure, for example the gel phase can be measured by a reduction in its viscosity that occurs upon dilution with water. For any given quantity of water, the extent of viscosity reduction is directly related to how quickly and easily it will be removed from the surface. The amount of water required to rinse a cosmetic composition from a surface is, therefore, directly related to the rate of viscosity reduction of the composition upon contact with water.

The measured viscosities are related to the rinse properties of the composition. For example, how quickly and how easily it will be removed from a surface. The lower the viscosity, the easier and quicker it will be removed from a surface. When it has been removed from the surface, the consumer will stop rinsing, thus preventing further consumption of water. This can, therefore, be correlated to the quantity of water required to rinse the composition from a surface.

Preferably, the surface is a hair surface.

When a conditioning gel phase composition is applied to hair during a wash/care process, the gel phase is deposited onto the hair surface. When the deposited gel phase comes into contact with water (during a rinse step), the structure of the gel phase must be broken up in order for it to be efficiently removed from the hair. This disruption to the gel phase affects its viscosity. Thus, a reduction in viscosity occurs upon dilution with water. The greater the disruption to the gel phase, the easier and faster it is removed and, ipso facto, the less water is required to complete the rinse. Thus, for any given quantity of water, the extent of viscosity reduction indicates how quickly and easily it will be removed from the hair. This correlates with the amount of water used to rinse a conditioning composition from hair.

### EXAMPLES

Embodiments of the invention will now be illustrated in the following examples.

### Example 1: Compositions A, B and C

The following hair conditioner compositions were prepared: —

TABLE 1
Composition of hair conditioner A, B and C

| | Amount (wt %) | | |
|---|---|---|---|
| Material (based on 100% active) | A | B | C |
| Behenyl Trimethyl Ammonium Chloride | 0.7 | 1.05 | 1.05 |
| Stearamidopropyldimethylamine | 0.7 | 1.50 | 1.50 |
| Cetearyl Alcohol | 4 | 6.0 | 6.0 |
| Acrylates/Beheneth-25 Methacrylate | 0.1 | 0.10 | 0.10 |
| Coploymer (Aculyn 28) | | | |
| Lactic acid | 0.35 | — | — |
| Stearic acid | — | 0.1 | — |
| Paraffin wax | — | 1.0 | — |
| Conditioning silicone | 1.5 | 3.0 | 3.0 |
| Fragrance/preservatives/water | To 100 | To 100 | To 100 |

The conditioners were prepared using the following methods:

Conditioner A
1. Water was added to a suitable vessel, lactic acid and the copolymer were added, and the vessel heated to 80° C.
2. Cetearyl alcohol was then added to the formulation along with tertiary amine salt (stearamidopropyldimethylamine).
3. At 80° C. the Behenyl Trimethyl Ammonium Chloride (BTAC) was added and the resultant mixture mixed until opaque and thick.
4. The heat was then turned off and the quench water was added.
5. The mixture was then cooled to below 40° C. the rest of the materials, including fragrance, were added.
6. Finally the formulation was mixed at high shear on a Silverson mixer at 5000 rpm for 5 minutes.

Conditioners B and C
Approximately 35% wt. of the water was heated to 65 to 70° C. prior to addition of stearmidopropyl dimethylamine with mixing until completely dissolved.
The polymer, Aculyn 28, was then added at high shear.
Separately, behenyltrimethyl chloride, cetearyl alcohol, stearic acid (and paraffin if present) were melted together and the resultant molten mixture added to the aqueous phase.
The rest of the water was added as mixture was cooled to 40 to 45° C. before the conditioning silicones, fragrances and preservatives were added.

### Example 2: Viscosity of Compositions a, B and C Under Dilution

In the following examples, viscosity measurements were carried out on aqueous dilutions of the neat compositions prepared above.

Samples were measured using a Brookfield viscometer with a T-A spindle as well as RVS.

The samples were prepared as 150 g dilutions as follows:

Composition (for example 75 g for a 1 in 2 dilution) was added to a beaker. Water (75 g for a 1 in 2 dilution) was then added in small amounts with mixing until homogeneous.

The sample was left to equilibrate for one hour before measurement with the Brookfield viscometer.

In this way, a series of dilutions were prepared (ensuring consistent mixing and speed of water addition throughout).

The samples were measured using the Brookfield RVDV-II+ viscometer with the following conditions: T-A bar spindle: 0.5 rpm; 60 s measurement; 5 replicates per sample.

The results are given in the following table:

TABLE 2
Viscosities of Compositions A, B and C

| | Viscosity/cP | | | Normalised data | | |
|---|---|---|---|---|---|---|
| Dilution | A | B | C | A | B | C |
| Neat | 628000 | 781600 | 628000 | 500000 | 500000 | 500000 |
| 1 in 1.25 | 159200 | 260000 | 197600 | 126751.6 | 166325.5 | 157324.8 |
| 1 in 1.5 | 82400 | 175200 | 193600 | 65605.1 | 112077.8 | 154140.1 |
| 1 in 1.75 | 47200 | 143200 | 156800 | 37579.62 | 91606.96 | 124840.8 |
| 1 in 2 | 21600 | 97600 | 144800 | 17197.45 | 62436.03 | 115286.6 |
| 1 in 3 | 1600 | 17600 | 58400 | 1273.885 | 11258.96 | 46496.82 |
| 1 in 4 | 800 | 5600 | 15200 | 636.9427 | 3582.395 | 12101.91 |

When a conditioning gel phase composition is applied to hair during a wash/care process, the gel phase is deposited onto the hair surface. When the deposited gel phase comes into contact with water (during a rinse step), the structure of the gel phase must be broken up in order for it to be efficiently removed from the hair. The greater the disruption to the gel phase, the easier and faster it is removed and, ipso facto, the less water is required to complete the rinse. The disruption to the composition gel phase is indicated by a reduction in its viscosity upon dilution with water.

For any given quantity of water, the extent of viscosity reduction indicates how quickly and easily it will be removed from the hair. This correlates with the amount of water used to rinse a conditioning composition from hair.

It will, thus, be seen that the rinse properties of the different compositions can be distinguished.

1. A method of predicting rinse properties of a composition from a surface, comprising the steps of:
i) providing a neat treatment composition;
ii) preparing a series of aqueous dilutions of the neat treatment composition;
iii) measuring the viscosities of the neat treatment composition and the aqueous dilutions of the treatment composition, using a suitable method such as a Brookfield viscometer fitted with a T-B spindle and Helipath, at 0.5 rpm and 25° C.;
iv) correlating the measured viscosities to the rinse properties of the neat treatment composition;
v) optionally correlating the rinse properties of the neat treatment composition to the amount of water used to rinse the neat composition from a surface.

2. The method as claimed in claim 1, wherein the composition is selected from a gel composition and a lamellar composition.

3. The method as claimed in claim 1, wherein the surface is a hair surface and the neat treatment composition is a neat hair treatment composition.

4. The method as claimed in claim 1, wherein the composition is a rinse off composition.

5. The method as claimed in claim 3, wherein the composition is a hair conditioner.

6. The method as claimed in claim 5 wherein the hair conditioner contains a gel phase.

7. The method as claimed in claim 1, wherein the neat treatment composition comprises a viscosity reduction agent.

8. The method as claimed in claim 6, wherein the viscosity reduction agent is a hydrophobically modified anionic polymer.

9. The method as claimed in claim 1, wherein from 2 to 8 aqueous dilutions are used.

10. The method as claimed claim 1, wherein the viscosity of the neat treatment composition is measured before and after the addition of a viscosity reduction agent.

11. The method as claimed in claim 1, comprising carrying out the method using a first neat treatment composition and then carrying out the method using a second neat treatment composition and comparing the viscosities of the first and second neat treatment compositions.

12. The method as claimed in claim 11, comprising repeating steps (i) to (iv) for a second neat treatment composition and comparing the viscosities of the first and second neat treatment compositions to determine the relative rate of rinsing of the first and second neat treatment compositions.

13. The method as claimed in claim 11, further comprising correlating the viscosity and/or rate of rinsing of the first and second neat treatment compositions to the amount of water used to rinse the neat composition from a surface.
